(19) United States
(12) Patent Application Publication
(10) Pub. No.: US 2024/0407924 A1
(43) Pub. Date: Dec. 12, 2024

(54) ENDOPROTHESES COMPONENT FOR JOINTS

(71) Applicants: BUDAPESTI MUSZAKI ES GAZDASAGTUDOMANYI EGYETEM, BUDAPEST (HU); SEMMELWEIS EGYETEM, BUDAPEST (HU)

(72) Inventors: GABOR SZEBENYI, VECSES (HU); GYORGY KOCSIS, BUDAPEST (HU); ZOLTAN KISS, BUDAPEST (HU)

(21) Appl. No.: 18/286,049

(22) PCT Filed: Apr. 6, 2022
(86) PCT No.: PCT/HU2022/050030

§ 371 Date: Oct. 6, 2023

(30) Foreign Application Priority Data
Apr. 8, 2021 (HU) P2100145

(51) Int. Cl. A61F 2/30

(52) U.S. Cl. CPC A61F 2/30771

(57) ABSTRACT

An improved device for converting a semi-automatic self-loading handgun into a fully automatic weapon. The handgun is fixed in the front part of a case. An operating trigger is arranged in the case behind the handgun. Between the operating trigger and the trigger of the handgun a means is provided that causes pulling the trigger in a repeated series as long as the operating trigger is pulled. A hitting element is locked when being in rear position and when the operating trigger is in non-pulled state. A rocking arm causes the pulling of the own trigger when the hitting element is forced by a spring to move forward in the pulled state of the operating trigger. A locking assembly is provided and prevents forward movement of the hitting element, this locking starts when the slide has moved into rearmost position until the slide returns into its forward base position.

The present invention relates to an articular endoprosthesis component for joints that can be fitted to a counterpart joint component to provide a friction-reduced connection between them. The invention further provides a pair of such components for arthroplasty.

Joints are connections between adjacent bones. In the case of healthy musculoskeletal systems, the cartilage surfaces of the joints are covered with plain glass cartilage, which allows the two bone ends to fit together seamlessly. The joint is covered outside by a closed joint shell, which produces the fluid needed for mobility, which acts as a kind of lubricant. During aging, and/or under significant joint stresses, diseases and lesions can develop in these areas, the only cure for which is often a surgical implantation of a joint endoprosthesis.

Solid-state prosthesis systems commonly used in orthopedics are characterized by the wear of the joint surfaces of the prosthesis, which, in addition to destroying the surface, also adversely affects the life of the prosthesis through its ability to cause bone resorption and loosening. Although joint fluid is always present around artificial components, hydrodynamic lubrication virtually never develops between the implanted solid metal or plastic components.

To overcome the above shortcoming of artificial prosthetic elements made of solid material, the literature mentions several approaches in which the displacement of the components on each other is addressed by surface boundary layer stabilization. In the case of these inventions, triangular, rectangular, or circular surface microstructures and recesses are typically formed on the contact surfaces of the prosthetic elements, by means of which the joint fluid can adhere by forming a surface boundary layer, thus reducing the friction between the components. The production of such prostheses is relatively simple also by generally known production technology methods, as it is sufficient to provide surface treatment and design only. However, the condition for formation of a reliable boundary layer is the constant motion reaching a certain speed level, which is not always ensured in the human joints, especially at rest and at the very beginning of each motion when they start.

Although one skilled in the art is not expected to consider remote engineering solutions such as general mechanical engineering, it is worth referring to the operation of lubricated porous sintered bronze bearings. The self-lubricating bearing is a porous metal component impregnated with lubricating oil. The oil in the pores provides continuous lubrication between the bearing and its rotating shaft, eliminating the need for additional external lubricating oil. Self-lubrication allows a sliding bearing to operate under hydrodynamic conditions, resulting in a very low coefficient of friction. However, in contrast to biological, i.e., human and animal systems, in engineering different motion conditions prevail, because here—due to the rapid rotation unimaginable in living systems—the contact of the elements disappears after a few turns, as the lubricant separates the friction surfaces as the speed increases and keeps the shaft floating. The porosity of that bearing is high (up to 20-25%), which is necessary for its lubricant storage function, but it must also be sufficiently strong, so sponge-like compression under load can occur, which confirms that it represents an irrelevant direction of development in the field of endoprostheses.

In earlier investigations, fatigue wear was observed on the components of shoulder prostheses, especially on their polyethylene surfaces, which resulted in particles detaching from the surface of the polyethylene insert in larger pieces and flakes. This confirms that improving lubrication to reduce joint wear appears to be a workable approach.

The goal of the development was to create a system that significantly improves lubrication between prosthetic components and reduces wear.

Of the artificial prosthesis systems, complete endoprostheses consist of a head and a glenoid component. In the lubrication system of the present invention, at least one of the two contact surfaces (e.g., the head component) is formed to have a porous cellular structure. The porous surface absorbs the joint fluid and delivers the lubricating joint fluid to all parts of the surface to be lubricated. In this way, the joint fluid, which serves as a lubricant, ensures the lubrication of the contact surfaces even during the mere contact of the components, i.e., even at rest and unloaded state. This is exacerbated when, during movement, the counterpart, which is usually solid, presses against the porous component, which compresses to some extent due to its flexibility, allowing more joint fluid to flow from its inner cells to the contact surfaces.

A material can be porous in several ways, and in one embodiment of the present invention, a Voronoy cell arrangement is used. Voronoy cells can be derived mathematically as well described in the literature. Such cells form a spongy structure. The other contact surface of the artificial joint is generally solid that ensures its strength when even polyethylene is used.

Our object is achieved by developing a joint endoprosthesis component which can be fitted to a joint counter-component, and which has a cell structure with a predetermined thickness on the surface to be fitted to the joint counter-component.

Preferably, the cell structure comprises Voronoy cells.

The cell structure preferably comprises first cells and second cells, wherein the characteristic dimension of the first cells is preferably from 5 μm to 100 μm; and the characteristic dimension of the second cells is preferably from 100 μm to 300 μm. The thickness of the cell structure is preferably between 0.5 and 10 mm.

Preferably, the cell structure is made of titanium alloy or medical steel.

Preferably, the material of the cell structure is selected from the group consisting of polyethylene, poly (ether-ether ketone).

The joint counterpart is preferably a joint endoprosthesis component.

The joint counterpart is preferably made of solid material.

The material of the joint counterpart component is preferably selected from the group consisting of the following polymers: polyethylene, poly (ether-ether ketone).

The invention will now be described in detail by reference to the accompanying drawings, in which:

FIG. 1 is an arrangement for a pair of components (glenoid head) in a general prosthesis,

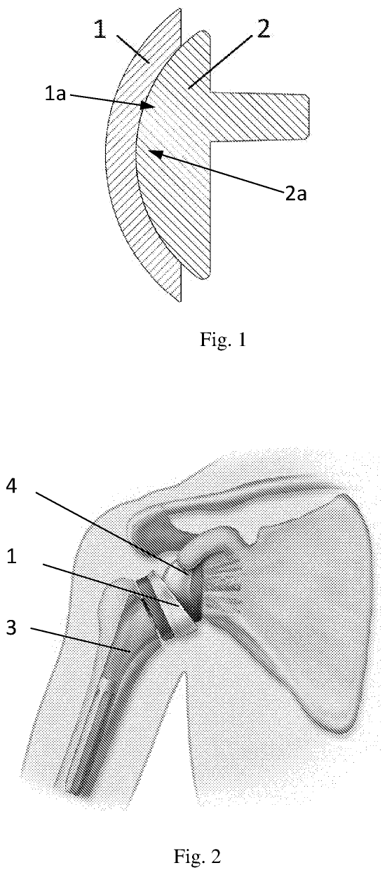

FIG. 2 shows a location of so-called inverse shoulder prosthesis in the patient's body (source: https://clevelandshoulder.com/reverse-total-shoulder-replacement-surgery/),

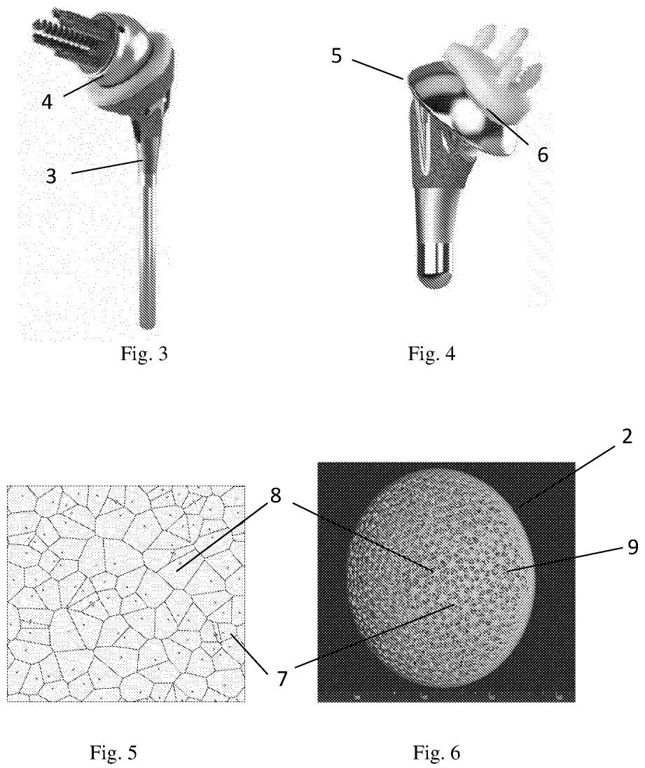

FIG. 3 is an image of an inverse shoulder prosthesis (source: https://www.djoglobal.com/products/djo-surgical/altivate-reverse),

FIG. 4 is an image of an anatomical shoulder prosthesis (source: https://www.djoglobal.com/products/djo-surgical/altivate-anatomic),

FIG. 5 is an illustration of the Voronoj principle on a planar point system (source: https://www.researchgate.net/figure/Voronoi-cells-on-a-2D-surface-Illustrated-is-the-capture-zone-areas-for-randomly_fig3_267400965), and

FIG. 6 is a head component with cells according to the invention.

Generally, each artificial joint consists of a socket 1 component and a head 2 component, which are illustrated by the component pair shown in FIG. 1. The head 2 is usually spherical and the socket 1 is a concave-shaped element at least partially accommodating the head 2. To illustrate the relative position of the prosthesis components 1, 2 and their location in the patient's body, and to distinguish between the names of the articular endoprosthesis components used in orthopedics, see a prior art shoulder joint endoprosthesis in FIGS. 2-4. This type of prosthesis is a good illustration of the two basic types of fitting of the contact shoulder prosthesis components 3, 4, 5, 6: the so-called reverse replacement, shown in FIGS. 2 and 3 and the so-called standard replacement as shown in FIG. 4. In the case of a shoulder, the bone segment containing the socket 1 is called glenoid 3. Considering the inverse shoulder prosthesis shown in FIGS. 1 and 2, the rounded piece, which should basically be called the head 2, is not in its original position, that is not on the upper end of the humerus, but on the glenoid 3. Therefore, since it was placed on the side of the original 1 socket, it is exceptionally not referred here to a head, but to a dome 4, to avoid confusion. FIG. 4 shows the head 5 and socket 6 of the standard (anatomical) shoulder prosthesis. It should be noted that an inverse prosthesis exists only for the shoulder. Hereafter, the reference to the head/socket components of the arthroplasty component of the present invention also includes the specific dome term used for shoulder prostheses.

The use of the arthroplasty components 1, 2 according to the invention is recommended mostly in an endoprosthesis system comprising a metal component and a polymeric counterpart component being capable of displacing on one another. Consequently, an articular endoprosthesis component 2, 1 according to the invention, which may be a head 2 component or a socket 1 component, can be fitted to another articular counterpart component 1, 2—to a socket 1 or to a head 2—and a cell structure 9 in a predetermined thickness is formed a surface 1a, 2a fitted to the articular counterpart component 1,2.

The thickness of the cell structure 9 is preferably between 0.5 and 10 mm, but this thickness may also reach the entire volume of the prosthesis component 1, 2. This is typically useful when the total thickness of the head 2 or socket 1 prosthesis component does not exceed 0.5 mm, or the flexibility is particularly relevant to the arthroplasty component of the joint. If the cell structure is formed in a part of the endoprosthesis component 1, 2 only and the rest of the volume of the component 1, 2 is formed as a solid material, the stability of the endoprosthesis component 1, 2, in addition to the flexible, porous structure, is also ensured.

The articular component 1,2 provided with a cell structure 9 on its contact surface is typically the head 2 component (FIG. 6), and it is advantageously made of a Ti alloy or medically approved steel. According to another embodiment, the component 1, 2 provided with the cell structure 9 is a socket 1 side component.

The articular counter-component is typically an artificial prosthesis component, too, and its full volume is made of solid material, preferably a polymer such as polyethylene or poly (ether-ether-ketone). In another combination, the counterpart component 2, 1 is also an endoprosthesis component provided with a cell structure 9 according to the invention.

However, there may be applications—such as the so-called hemiarthroplasty—, where the component 1, 2 with a cell structure 9 according to the invention is not associated with another artificial counter-component 2, 1, but can be incorporated into the patient's body alone.

The design of the cell structure 9 can be realized by computer 3D modelling and then by 3D printing of the constructed model (mainly with DMLS technology). After implantation, synovia penetrates the microcavities of cell structure 9 due to joint pressure conditions and capillary action, and then has a beneficial effect during the movement of the patient.

The cells are preferably Voronoy cells, which can be used to create a permeable cell structure 9, that is a network of cells, which, in addition to the appropriate cell formation, can also reduce the apparent modulus of elasticity (structural rigidity) of the material, thereby improving lubrication. The inventive perception, that the articular endoprosthesis component 1, 2 has at least a partially porous cell structure 9 complemented by the Voronoj cell design, has the advantage that this cell structure is highly approaching a randomly built natural pattern, and thus a cell structure 9 easily adaptable to 3D shapes can be obtained by simple planning.

To form the Voronoy cells, the cell structure 9 can be made according to the following design principle, which is illustrated by means of the planar point system shown in FIG. 5, which can be transferred to a spatial point system in an analogous manner. In general, irregularly arranged points are allocated in a plane (or in the case of the present invention: in space). A polygon can be constructed around each point whose interior points (all its points except points that make up its boundary) are closer to the point in question than to all other points. Polygons (polyhedra) having this trait are convex and fill the plane (space) continuously. The sides of the polygon (the sides of the polyhedron) are perpendicular to the lines connecting the point with the other points and bisect them. (Source: Dr Ferenc Sárközi, http://www.agt.bme.hu/tutor_h/terinfor/t27.htm).

In the case of a regular grid of points the Voronoy cell formation transfers the grid of points to a regular mosaic grid of cells. The advantage of Voronoy cells is that the degree of elasticity of the porous surface can be well adjusted during production based on the thickness of the cell walls forming the cell structure 9 as a sponge, and the size of the pores (cell size). In the present invention, the spatial point distribution is preferably arranged so that essentially two cell sizes can be created. The characteristic dimension of the first cells 7 having a smaller cell size, which is usually the largest diagonal or the largest side edge, preferably ranges from 5 μm to 100 μm, while the characteristic dimension of the second cell 8 with a larger cell size is preferably from 100 μm to 300 μm. In this way, it is possible for the synovia (articular fluid) to flow into the first cells 7 due to the capillary action; and any particles (approximately 20 μm in size) are trapped in the second cells 8 so that they do not migrate off the friction surfaces 1a, 2a.

The subject of the patent application can be used for any type of joint, such as a shoulder, elbow, thumb, ankle, knee, or even hip prosthesis; Thus, it can appear in joint implants produced by the industry of medical instruments regardless of the type of joint implant, practically.

Due to the cellular design, the modulus of elasticity of the porous joint endoprosthesis component 1,2 is such that it can adapt well to the surface of the opposing component, even if the counter component 2,1 is made of polymer; thus, ensuring that the articular fluid stored in the cells is squeezed out and reaches the appropriate point of contact.

By means of the invention, the articular element(s) having a cell structure 9 according to the invention ensures that a part of the articular fluid remains between the friction surfaces 1a, 2a even in unloaded state, thereby reducing friction and wear, increasing the service life of the joint implant, and likelihood of an immune response activated by abrasion products is reduced.

1. An articular endoprosthesis component (1, 2) for joints having a friction surface (1a, 2a), which can be fitted to the friction surface of a counter component (1,2) of said joint, characterized in that a cell structure (9) having a predetermined thickness is formed on the friction surface (1a, 2a) to be fitted to a counter component of the joint.

2. The articular endoprosthesis component (1, 2) for joints according to claim 1, wherein the cell structure (9) comprises Voronoj cells.

3. The articular endoprosthesis component (1, 2) for joints according to claim 1, wherein the cell structure (9) comprises first cells (7) and second cells (8), wherein characteristic dimension of the first cells (7) is preferably in a range from 5 μm to 100 μm; and the characteristic dimension of the second cells (8) is preferably from 100 μm to 300 μm.

4. The articular endoprosthesis component (1, 2) for joints according to claim 1, wherein the cell structure (9) has a thickness of a range between 0.5 mm and 10 mm.

5. The articular endoprosthesis component (1, 2) for joints according to claim 1, wherein it is made of titanium alloy or medical steel.

6. The articular endoprosthesis component (1, 2) for joints according to claim 1, wherein it is made of a material selected from the group consisting of polyethylene and poly (ether-ether ketone).

7-9. (canceled)
